(19) United States
(12) Patent Application Publication
(10) Pub. No.: US 2018/0168727 A1
(43) Pub. Date: Jun. 21, 2018

(54) ELECTROMAGNETIC WAVE THERAPY DEVICE AND IMMUNOTHERAPY METHOD THEREOF

(71) Applicant: MR. LOOP CO., LTD., New Taipei City (TW)

(72) Inventors: CHIH-MIN LIN, NEW TAIPEI CITY (TW); YA-CHUNG YU, NEW TAIPEI CITY (TW)

(21) Appl. No.: 15/840,242

(22) Filed: Dec. 13, 2017

Related U.S. Application Data
(60) Provisional application No. 62/435,185, filed on Dec. 16, 2016.

(51) Int. Cl. A61B 18/18; A61N 5/06; A61N 2/00; A61N 1/02

(52) U.S. Cl. CPC A61B 18/18; A61N 5/062; A61N 2005/1074; A61N 1/025; A61N 2005/0627; A61N 2/002

(57) ABSTRACT

The present invention provides an electromagnetic wave therapy device and an immunotherapy method thereof, which sense an ambient temperature and a body temperature of a patient by a sensing unit, thereby generating a sensing signal. A control unit, based on indication information, sensing signals and finding a corresponding spectrum indication signal in the database. The baseband unit in the treatment device generates the therapeutic spectrum content corresponding to different waveforms, frequencies, amplitudes or timetable according to the therapeutic spectrum content, and then uses a radio frequency unit emitting a millimeter-wave at a frequency between 56 GHz and 65 GHz to become a millimeter-wave irradiation source, radiates a patient with a desired location to be treated, and allows the patient to perform a millimeter-wave immunotherapy program by radiating the millimeter-wave treatment device with a non-invasive way to achieve the body's immune cells performance of the purpose and to control and change the human immune system.

### REFERENCE TO RELATED APPLICATION

This Application is based on Provisional Patent Application Ser. No. 62/435,185, filed 16 Dec. 2016, currently pending.

### FIELD OF THE INVENTION

The present invention relates to an electromagnetic wave therapy device and an immunotherapy method thereof. More particularly, the present invention relates to a millimeter-wave therapy device using frequencies between 56 GHz and 65 GHz and capable of providing multi-site usage and an immunotherapy method thereof.

### BACKGROUND

In recent years, due to medical technology has been greatly improved, electromagnetic waves and/or light waves are broadly used to treat various diseases, of which the most attention is given to the millimeter-wave therapy. Millimeter-wave therapy is mainly through the specific frequency of millimeter-waves and human cells produce resonance phenomenon, so that the human body cells return to normal conditions, in order to achieve the regulation of physiological function, relieve symptoms, improve immunity, treatment and improvement and other effects.

Conventional millimeter-wave therapy devices can emit only a single fixed millimeter-wave frequency. When there are different frequency needs, only millimeter-wave therapy devices of different frequencies to be purchased in order to meet the requirements for the user. In addition, the medical treatment of the millimeter therapy device, due to its large size and difficult to carry, patients must be treated under the guidance of medical staffs and can not carry around or use in long distance. Therefore, patients often need to run around, greatly reducing the convenience of millimeter-wave therapy device application.

For this reason, the present invention provides a millimeter-wave therapy device and an immunotherapy method thereof, as solutions to the above problems.

### SUMMARY

The major object of the present invention is to provide a millimeter-wave therapy device which can design and fix a plurality of components of a millimeter-wave therapy device in a millimeter-wave therapy chip and transmit an instruction to provide a millimeter-wave radiation source of 56 GHz to 65 GHz for allowing the patient to carry out millimeter-wave treatment procedures, but also at any time to adjust the millimeter-wave radiation data, to achieve the best therapeutic effect.

Another object of the present invention is to provide a millimeter-wave therapy method which can download a corresponding spectrum indication signal from a database according to the disease item to be treated and the patient's temperature and other symptom data, the baseband unit generates therapeutic spectrum contents corresponding to different waveforms, energies, frequencies, pulses, amplitudes and different timetables according to the spectrum indication signals, and generates millimeter-wave radiation sources via the radio frequency unit to not only radiate and treat the skin of the muscles, nerves or internal organs, but also to update the database and improve the treatment effect.

Another object of the present invention is to provide a millimeter-wave therapy device which can store the spectrum indication signal in a memory unit in the millimeter-wave therapy device or directly download from the cloud database via the communication device to achieve miniaturization, portable, programmable and low-power millimeter-wave therapy devices.

Another object of the present invention is to provide a millimeter-wave therapy device and an immunotherapy method, which can be used in combination with indications for medication to reduce the inflammatory index, reduce the indicator of an oxidative stress, adjust the immunity and reduce the progression of the tumor. The purpose of the human body lesions or abnormal cells returned to normal state, to achieve the regulation of physiological function, soothing symptoms, treatment and improvement and other effects.

Another object of the present invention is to provide a millimeter-wave therapy device and an immunotherapy method for providing a relatively simple and convenient treatment for patients who need to go to and from the hospital without having to travel by car and doctors can also adjust the control through a server instruction or monitor the results of millimeter-wave therapy records to control patient's treatment status remotely.

To achieve above objects, the present invention provides an electromagnetic wave therapy device comprising: a control unit providing a first control instruction; a sensing unit, electrically connected to the control unit, for generating a sensing signal; a memory unit, electrically connected to the control unit, for storing at least one data reference table, wherein the data reference table records a spectrum indication signal corresponding to the sensing signal; a baseband unit electrically connected to the control unit for generating a corresponding therapeutic spectrum content according to the spectrum indicating signal; and a radio frequency unit electrically connected to the control unit for emitting a millimeter-wave at a frequency between 56 GHz and 65 GHz and combining with the therapeutic spectrum content to become a millimeter-wave radiation source.

To achieve above objects, the present invention provides an immunotherapy method using the electromagnetic wave therapy device, comprising following steps: confirming or input an indication of information; detecting an ambient temperature or a body temperature of a patient to generate the sensing signal; a control unit reading a sensing signal and indication information and finding a corresponding spectrum indication signal in a data reference table; controlling a baseband unit to generate a therapeutic spectrum content according to a spectrum indication signal; controlling a radio frequency unit to generate a millimeter-wave with a frequency between 56 GHz and 65 GHz and making it a millimeter-wave radiation source in combination with the content of the therapeutic spectrum content; the millimeter-wave radiation source illuminating at least one indication area.

In one embodiment of the present invention, wherein the control unit, the baseband unit, the radio frequency unit and the memory unit are integrated in a single bio-chip to become a millimeter-wave therapeutic chip.

In one embodiment of the present invention, wherein the data reference table comprises an indication information, a plurality of cytokines parameters or an oxidative stress index parameter, and the sensing signal may correspond to the indication information, the plurality of cytokines or the oxidative stress index parameters and there is a corresponding of the spectrum indicating signal.

In one embodiment of the present invention, wherein the control unit is connected with a second control device, and the second control device can provide the data reference table, the spectrum indication signal or a second control instruction.

In one embodiment of the present invention, comprising: a receiving unit, connected to the control unit and the second control device, for receiving the second control instruction or the data reference table; and a transmitting unit, connected to the control unit and the second control device, for transmitting the sensing signal or the data reference table to the second control device.

In one embodiment of the present invention, comprising: an antenna unit electrically connected to the radio frequency unit for focusing and transmitting the millimeter-wave radiation source.

In one embodiment of the present invention, comprising: a display unit, electrically connected to the control unit, for displaying the sensing signal, the data reference table or the spectrum indicating signal; an input interface electrically connected to the control unit; and an expansion unit, electrically connected to the control unit, is a communication device or a data transmission device.

In one embodiment of the present invention, comprising an integrated database, electrically connected to the second control device for storing the data reference table.

In one embodiment of the present invention, wherein the data reference table comprises a timetable of emission parameter data, a waveform parameter data, a frequency parameter data, an energy parameter data, a pulse parameter data, an indication data, a sensing signal data, a plurality of cytokines parameter data, an oxidative stress index parameter, a spectrum indication signal and a combination of the above components.

In one embodiment of the present invention, comprising a medication with the indication.

In one embodiment of the present invention, comprising: transmitting the sensing signal to a second control device; the second control device further finding the corresponding spectrum indication signal from the sensing signal and the indication information; and transmitting the spectrum indication signal or the data reference table to the control unit.

In one embodiment of the present invention, wherein the spectrum indication signal or data reference table is stored in the memory unit, and the data reference table comprises: a timetable of emission parameter data, a waveform parameter data, a frequency parameter data, an energy parameter data, a pulse parameter data, an indications data, a sensing signal data, a plurality of cytokine parameter data, an oxidative stress index parameter or spectral indication signal and other data.

In one embodiment of the present invention, comprising: the millimeter-wave radiation source being radiated with the indication area through the antenna unit comprising:
the millimeter-wave radiation source being radiated with the indication area through the antenna unit.

In one embodiment of the present invention, comprising: the second control device generating a plurality of spectrum indication signals; transmitting the plurality of spectrum indication signals to the control unit and generating corresponding millimeter-wave illumination sources according to each of the spectrum indication signals; and through the instruction of the second control device, radiating the patient each millimeter-wave radiation source to a corresponding different indication area.

In one embodiment of the present invention, comprising: collecting and analyzing the radiation course record, treatment result, millimeter-wave sample data or sensing signal of the millimeter-wave radiation source, and then adjusting the spectrum indication signal.

The characteristic, the implementation and efficacy, and the drawings for the best embodiments of the present invention are described in detail later.

### BRIEF DESCRIPTION OF THE DRAWINGS

FIG. 1 is a view of the present invention electromagnetic wave therapy device according to preferred embodiment.

FIG. 2A to 2C are diagrams of therapeutic spectrum content and millimeter-wave radiation sources generated by a baseband unit according to various embodiments of the present invention.

FIG. 3 is a structural view of another embodiment of the electromagnetic wave therapy device of the present invention.

FIG. 4 is a structural view of another embodiment of the millimeter-wave therapy device of the present invention.

FIG. 5 is a flowchart of the operation of a preferred embodiment of the electromagnetic wave therapy method of the present invention.

FIG. 6 is a flowchart of the operation of the electromagnetic wave treatment method according to still another embodiment of the present invention.

FIG. 7, FIG. 8 and FIG. 9 are the experimental data of the electromagnetic wave therapy method of the present invention respectively after radiation experiment and the control group on the TNF-α, COX-2, IL-6 results.

### DETAILED DESCRIPTION OF THE PREFERRED EMBODIMENTS

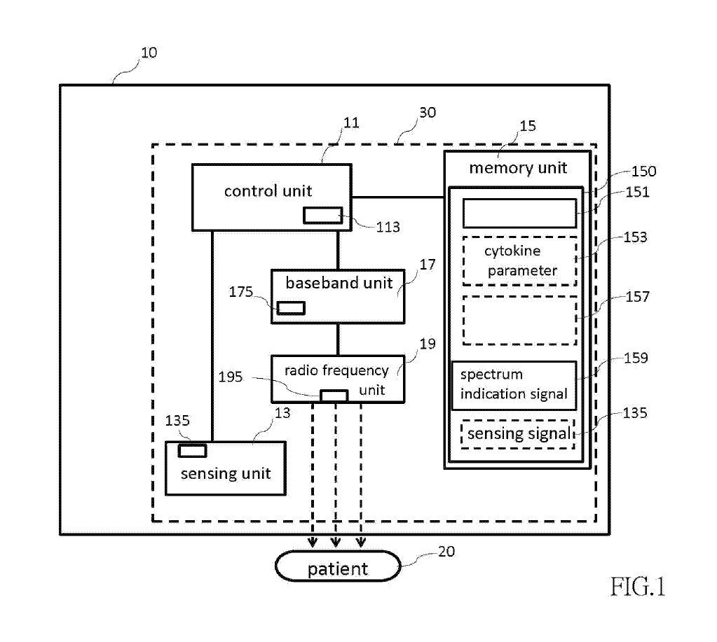

Referring to FIG. 1, which is a structural view of a preferred embodiment of an electromagnetic wave (millimeter-wave) therapy device according to the present invention. As shown in the figure, the millimeter-wave therapy device 10 mainly includes a control unit 11, a sensing unit 13, a memory unit 15, a baseband unit 17 and a radio frequency unit 19. The control unit 11, the sensing unit 13, the memory unit 15, the baseband unit 17, and the radio frequency unit 19 may be integrated in a single biochip such as a millimeter-wave therapeutic chip 30. The control unit 11 is electrically connected to the sensing unit 13, the memory unit 15, the baseband unit 17 and the radio frequency unit 19 respectively, and provides a first control instruction 113.

The sensing unit 13 may detect a symptom data such as an ambient temperature and/or a body temperature of a patient and generate a sensing signal 135 accordingly. The memory unit 15 is controlled by the control unit 11 and may store or temporarily store one or more data reference tables 150. The memory unit 15 may include but is not limited to: indication information (to treat the condition) 151, a plurality of cytokine parameters 153, (for example, including but not limited to TNF-α, IL-4 and IL-8, etc.) and/or parameters of an oxidative stress indicator 157 (e.g. ROS, NOS, COX1, COX2, NO and SOD, A spectrum indication signal 159 corresponding to each of the sensed signal 135, the indications 151, the plurality of cytokine parameters 153, and/or the oxidation pressure indicator 157 is described.

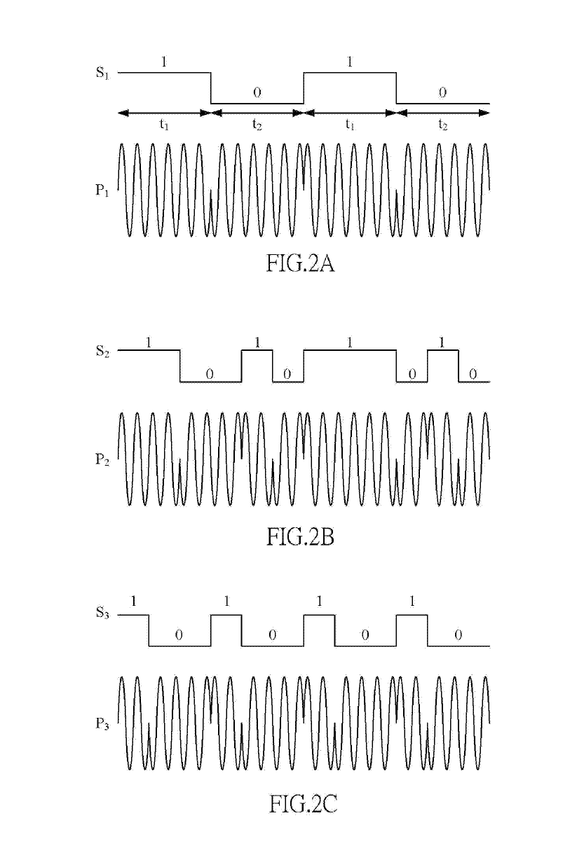

The baseband unit 17 is controlled by the control unit 11, receives the spectrum indication signal 159 from the memory unit 15, and generates a therapeutic spectrum content 175 accordingly. The therapeutic spectrum content 175 is composed of a time-domain parameter, a waveform parameter, a frequency parameter, an energy parameter, a pulse parameter, an amplitude parameter and/or a combination of sensing parameters. In other words, for different sensing signals 135, the plurality of cytokine parameters 153, an oxidative stress index parameter 157, and/or different indications 151 may be used to generate corresponding individual waveforms, time domains, frequencies, energies, pulsed and/or amplitude therapeutic spectral content 175, the fundamental frequency digital phase control signals S1, S2 and S3 shown in FIGS. 2A-2C.

Taking FIG. 2A as an example, the therapeutic spectrum content 175 generated by the baseband unit 17 is a baseband digital phase control signal S1, which can control the time domain phase of the analog electromagnetic wave in the space and the effect of the digital signal state 1 the time t1 and the action time of the digital signal state 0 are t2, and the fundamental frequency digital phase control signal S1 will generate a relative response to a specific immune cell biological factor.

The radio frequency unit 19 is controlled by the control unit 11 to receive the therapeutic spectrum content 175 of the baseband unit 17 and can be used as a carrier of the therapeutic spectrum content 175 to transmit a millimeter-wave radiation source 195 with a frequency of 56 GHz to 65 GHz. 2A to the millimeter-wave analog signals P1, P2 and P3 shown in FIG. 2C. The millimeter-wave radiation source 195 can radiate various parts of the body (indications area) of a patient or a user 20 for the purpose of being easy to carry and use.

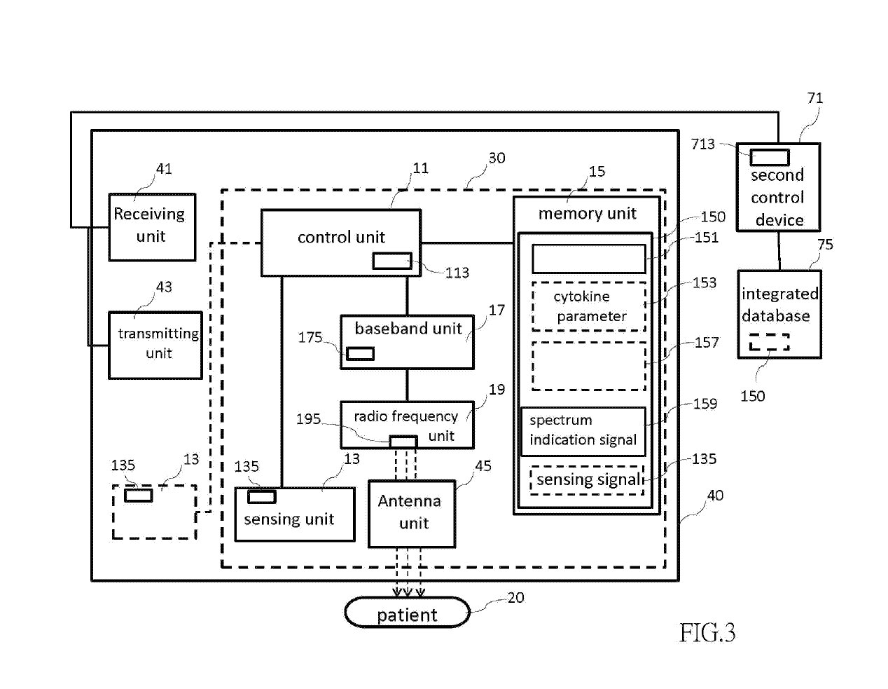

In addition, referring to FIG. 3, which is a structural view of another embodiment of the electromagnetic wave treatment device of the present invention. As shown in the figure, in this embodiment, the millimeter-wave therapy device 40 of the present invention may also be a module design, which includes a receiving unit 41 and a transmitting unit 43 in addition to the millimeter-wave therapy chip 30, and is electrically connected the control unit 11 is provided in the millimeter-wave therapy chip 30. The receiving unit 41 may receive the second control instruction 713 from a second control device 71. The second control device 71 is disposed outside the millimeter-wave therapy device 40. Through the second control instruction 713, the millimeter-wave radiation source 195 can be generated by the radio frequency unit 19 that controls the millimeter-wave therapy device 40 from outside or remotely. The second control device 71 also controls and retrieves a data reference table 150 from an integrated database 75 and temporarily stores the data reference table 150 in the memory unit 15.

The millimeter-wave therapy device 40 may also transmit the data reference table 150 stored in the memory unit 15 to the data reference table 150 stored in the integrated database 75 or updated in the integrated database 75 via the transmission unit 43. The integrated database 75 is electrically connected to the second control device 71. The doctor or professional instructor can read the data reference table 150 of the integrated database 75 through the second control device 71 and send it for analysis, the second control instruction 713 controls the millimeter-wave therapy device 40 remotely. In addition, the healthcare personnel can also adjust the spectrum indication signal 159 in the data reference table 150 in real time through the second control instruction 713 in response to the improvement or relief of the indication of the use side, and the spectrum indication signal 159 can be controlled, stored or temporarily stored in the integrated database 75 and/or the memory unit 15.

Further, in another embodiment of the present invention, the millimeter-wave therapy device 40 further includes an antenna unit 45 electrically connected to the radio frequency unit 19. The antenna unit 45 may be allocated inside the millimeter-wave therapy chip 30 or outside the millimeter wave therapy chip 30, and may be a horn antenna element, a waveguide antenna element, or an array antenna element for transmitting and focusing the millimeter-wave radiation source 195 generated by the radio frequency unit 19 collects the millimeter-wave radiation source 195 into a bundle, and the millimeter wave radiation source 195 can be controlled to radiate a deeper location of the body.

Via the receiving unit 41 to cause the control unit 11 to receive the second control command 713 of the second control device 71, the radio frequency unit 19 provides the millimeter-wave radiation source 195, which will radiate a patient 20, In particular, the body part of the patient 20, for example, a knee, a joint, a skin, and the like. In addition, the millimeter-wave radiation source 195 can also be used for inducing apoptosis in a tumor, increasing phagocytic activity of macrophages, increasing T cell proliferation and increasing B lymphocyte number, and the present invention is not particularly limited to Single-site treatment. The general millimeter wave system is an extremely high frequency electromagnetic wave with a frequency of 30 GHz to 300 GHz and a wavelength of 1 mm to 10 mm. The present invention uses a millimeter-wave of a frequency of 56 GHz to 65 GHz.

In another embodiment of the present invention, the sensing unit 13 senses the ambient temperature at which the device is located and the physiological signal of the patient 2 such as temperature, heartbeat, blood pressure and/or blood lipid concentration, etc. to obtain one or more sensing signal 135. The sensing signal 135 is controlled by the control unit 11 and stored in the memory unit 15.

In another embodiment of the present invention, the sensing signal 135 generated by the sensing unit 13 may also be transmitted to the second control device 71 via the transmitting unit 43, and the second control device 71 may further generate the sensing signal 135 based on the sensing signal 135 and/or indication information to the integrated database 75 to find the corresponding data reference table 150 and/or the spectrum indication signal 159. The second control device 71 further transmits the data reference table 150 and/or the spectrum indication signal 159 to the control unit 11 for temporary storage in the control unit 11 or the storage unit 15. The control unit 11 sends the first control instruction 113 to control the baseband unit 17 to generate the therapeutic spectrum content 175 and the RF unit 19 to generate the millimeter-wave radiation source 195. In another embodiment, the second control device 71 may be a medical staff or a treatment unit.

In still another embodiment of the present invention, the control unit 11 may also generate a plurality of second control instructions 713 according to the second control command 713, so that the plurality of second control instructions 713 provide different spectrum indication signals 159 Of the millimeter-wave radiation source 195 so that the patient can perform millimeter-wave therapy procedures in different frequency bands. For example, the “dosage” of the millimeter-wave for treating tumors of the joint or deep tissue may be different and the corresponding radiation times will be different. In these procedures, the second control instruction 713 is transmitted through the control unit 11, the baseband unit 17 and the radio frequency unit 19 to generate different millimeter-wave radiation sources 195. The user then moves the millimeter-wave therapy apparatus 40 to the site to be radiated to listen to the instruction from the medical staff or the treatment unit for performing different frequency, waveform, time domain, energy, pulse and/or amplitude millimeter wave irradiation program.

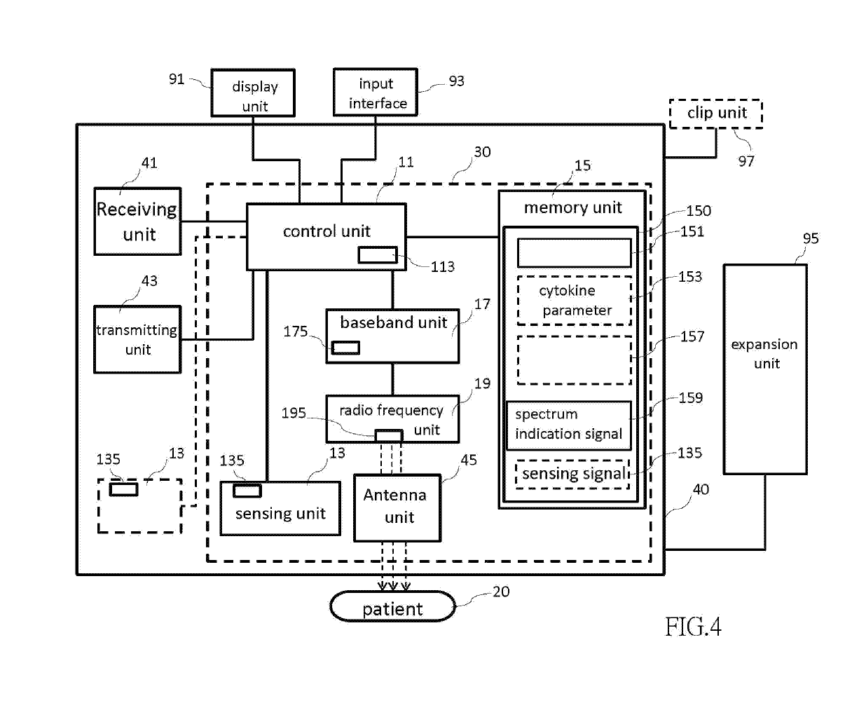

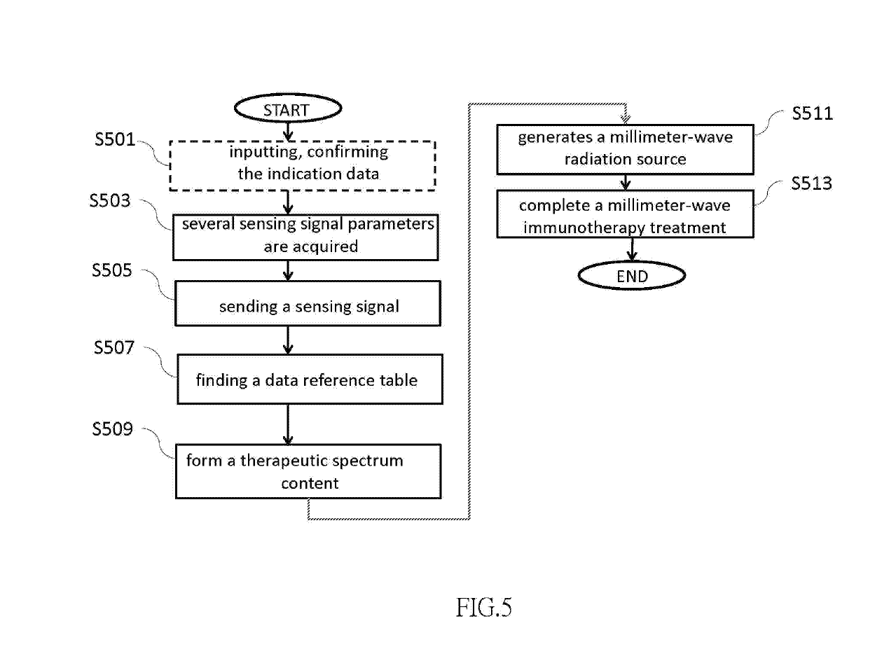

Referring to FIG. 4, which is a structural view of another embodiment of the millimeter-wave therapy device of the present invention. As shown in the figure, the millimeter-wave therapy apparatus 40 of the present invention may further include a display unit 91 and/or an input interface 93 electrically connected to the control unit 11. The display unit 91 may be used for displaying the sensing signal 135. The data reference table 150 and/or the spectrum indication signal 159 and other information. In addition, the input interface 93 of the further embodiment of the millimeter-wave therapy device 40 according to the present invention may be input for a voice or touch (key) mode for inputting the second control instruction 713 or the data reference table 150. The millimeter-wave therapy device 40 of the present invention may further include an expansion unit 95 electrically connected to the control unit 11 and may be a communication element (wired or wireless) or a data transmission element (Bluetooth, WIFI, etc.). For example, Through the expansion module 95, the millimeter-wave therapy apparatus 40 can be easily connected to a mobile phone, a portable electronic device, a computer, or the like. In addition, the expansion unit 95 can also select a power supply unit to provide external power to the millimeter wave therapy apparatus 40. The millimeter-wave therapy device 40 of the present invention may further include a clipping unit 97 to fix the millimeter wave therapy device 40.

Referring to FIG. 5, which is a flowchart of the operation of the electromagnetic wave therapy method according to a preferred embodiment of the present invention. As shown in the figure, the operation method of using the millimeter-wave therapy device 40 of the present invention includes the following steps: Step S501: inputting, confirming the indication data or performing proofreading on the indication data. In step S503, several sensing signal parameters are acquired, for example, temperature, blood pressure, heartbeat or blood lipid concentration. Step S505: sending a sensing signal to the control unit. Step S507, finding a data reference table and/or a spectrum indication signal corresponding to indications information and sensing signals, for example, but not limited to, transmitting a time domain parameter data, a waveform parameter data, a frequency parameter data, an energy parameter data, pulse parameter data, amplitude parameter data and/or sensing parameter data. In step S509, the baseband unit 17 processes the spectrum indication signal 159 and the modulation parameter conversion signal to form a therapeutic spectrum content 175. In step S511, the radio frequency unit generates a millimeter-wave radiation source. Step 513: Performing a patient irradiation with a millimeter-wave radiation source to complete a millimeter-wave immunotherapy treatment.

Furthermore, in an embodiment of the present invention, if the millimeter-wave therapy device of the present invention is a single disease treatment, the foregoing step S501 may also be omitted and not performed.

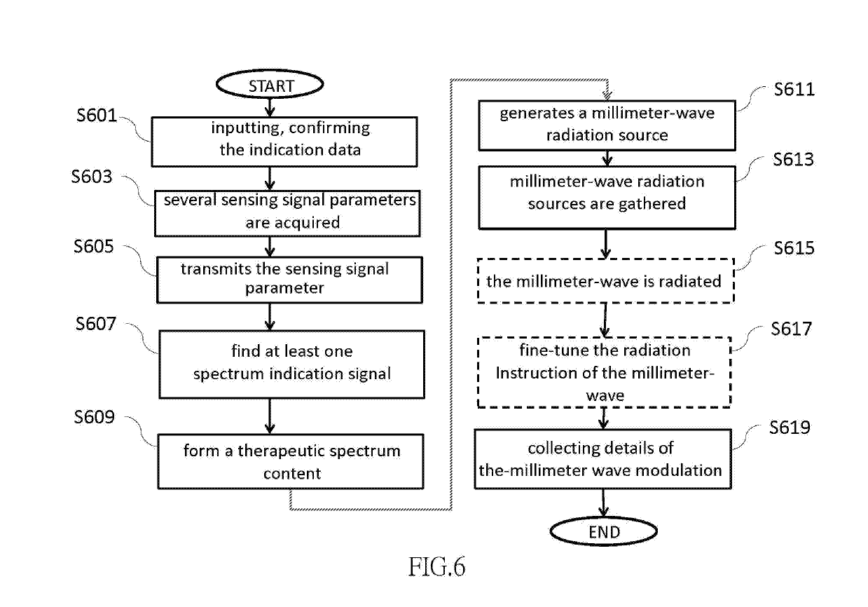

Referring to FIG. 6, which is a flowchart of another embodiment of the immunotherapy method of the present invention. The method for immunotherapy of the present invention comprises the following steps: Step S601, inputting, confirming indication information or proofreading the indication information to confirm that the condition to be treated is correct. In step S603, the sensing unit detects and acquires several sensing signal parameters, such as ambient temperature, or body temperature, blood pressure, heartbeat and/or blood lipid concentration, and the like. Step S605: the control unit transmits the sensing signal parameter to the second control unit. Step S607: The second control unit finds at least one spectrum indication signal and/or parameter reference table corresponding to the second control unit, and transmits the parameter reference table and/or the spectrum indication signal to the control unit. Step S609: The baseband unit is controlled by the control unit, and generates the therapeutic spectrum content according to the spectrum indication unit. In step S611, the radio frequency unit is controlled by the control unit to generate a millimeter-wave radiation source including the therapeutic spectrum content. In step S613, the millimeter-wave radiation sources are gathered into a bundle by the focusing action of the antenna unit, to increase the body-implantable depth of the millimeter-wave radiation source, for example, a tumor located in a non-surface tissue. To complete the millimeter-wave immunotherapy treatment by executing the steps descripted above.

In still another embodiment of the present invention, in step S615, the millimeter-wave is radiated to the indication area (body location), such as the skin or the joint, according to the location to be treated. Step S617: The control unit and/or the second control device and the chamber receive the sensing signal to fine-tune the radiation instruction of the millimeter-wave and to process the therapeutic spectrum content or the millimeter-wave radiation source in real time. In step S619, collecting details of the-millimeter wave modulation, the record of the millimeter wave radiation procedure, the condition of use, and the result of the treatment (information after treatment for relieving or ameliorating the symptoms such as the rate of tumor cell proliferation after radiation, etc.) Wave sample data, sensing signal and other data, and under the supervision of medical staff to adjust the spectrum indication signal or parameter reference table, and stored in the memory unit, the second control device or integrated database, the use of such data analysis and statistics, provide millimeter-wave emission modulation instructions for optimal therapy.

In another embodiment of the present invention, step S613 may be omitted. For example, the treatment location is a shallow area such as a skin, and antenna unit is not required.

In still another embodiment of the present invention, the spectrum indication signal 159 may include millimeter-wave sample data and may be stored in the memory unit 15. The spectrum indication signal 159 may also be electrically or communicatively connected through the transmitting unit 43 to transmit the spectrum indication signals 159 and/or the data reference table 150 to the integrated database 75. The collection system of the spectrum indication signal 159 comprises the following data which are given by way of illustration only, and thus are not limitative of the present invention, and which are: timetable of emission parameter data, waveform parameter data, frequency parameter data, energy parameter data, pulse parameter data, indication data, sensing signal data, pressure indicator parameters and/or spectrum indication signals and so on.

These spectrum indication signals 159 are stored in the integrated database 75 and can be utilized again for analysis. In addition, the timetable of emission parameter data, waveform parameter data, frequency parameter data, energy parameter data and pulse parameters (hereinafter referred to as millimeter-wave comprehensive modulation parameters), for example, the same disease A, at different locations of a body or in the depth of the tissue, the millimeter-wave settings are also slightly different. Because of the different shades of location, the millimeter-wave of the same “dosage” needs to be slightly adjusted for the duration of the radiation. Therefore, data is collected and analyzed through the integrated database 75. After calculation, send feedback to the millimeter-wave therapy device users or medical staffs.

The millimeter-wave therapy device of the present invention and its immunotherapy methods can also be used in combination with chemotherapy medicine. The cancer chemotherapy medicine (such as but not limited to 5-FU, Oxaliplatin and the like) that can be used in combination with the present invention. It is merely administered through transmit millimeter-wave radiation, cancer chemotherapy medicine inhibits tumor cell proliferation, activity and inhibition of oncoprotein performance, the data results performed well comparing with the control group. In addition, cancer chemotherapy with the use of the present invention, significantly improves inhibitory effect of tumor proliferation.

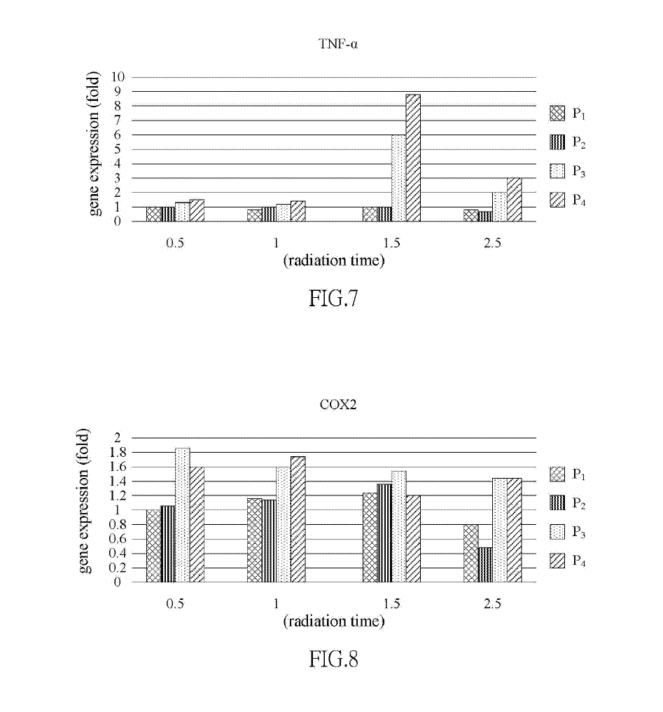

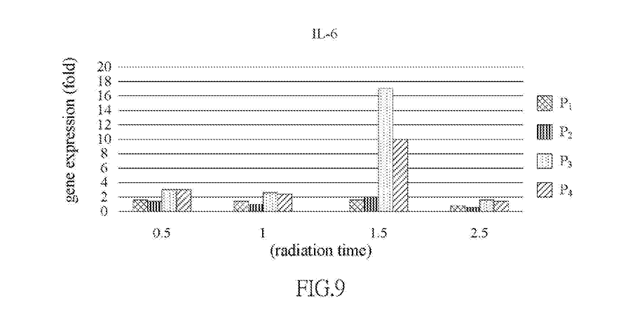

Eventually, FIG. 7, FIG. 8 and FIG. 9 are shown the experimental data of the electromagnetic wave therapy method of the present invention respectively after radiation experiment and the control group on TNF-α, IL-2, IL-6, IL-12α, IL-1β, Rel A and Cox 2. In one embodiment of the present invention, four types of P1, P2, P3 and P4 generated by using the millimeter-wave therapy device (10) of the present invention are respectively set using P1, P2, P3 and P4 millimeter-wave emission settings and radiate the same kind of immune cell line (for example, but not limited to, macrophages, killer cells or T cells, etc.), that is, human monocytic cell line THP-1 cells. 1×10⁶ THP-1 cells were cultured in 2 ml of culture solution (3.5 mm petri dish) for 24 hours, induced with PMA (150 nM) for 24 hours, and stimulated with LPS (10 pg/ml) for 4 hours. Next, a plastic pot is filled with about 1 cm depth of water and a stage is placed in the center so that the cell petri dish can be placed thereon. The radio frequency unit (19) of the millimeter-wave therapy device (10) needs to be set higher than the cell petri dish 10 cm at this time radio frequency unit (19) launch coverage area size of about 7 cm radius of the circle, all Petri dishes to be placed within this coverage.

Four millimeter-wave radiation sources (P1, P2, P3 and P4) with a radiation power between 0.5 W and 10 W were prepared for experiment. The experimental groups were radiated for 0.5 hr, 1 h, 1.5 hr and 2.5 hr, and at the point receive cells and culture solution. On the contrary, for the group of cells that do not radiate millimeter-waves, cells and culture solution are also collected at each time point.

RNA was extracted from the cells by using the RNeasy mini kit and DNase protocol, followed by reverse transcribing the extracted RNA into cDNA using the transcriptor first strand cDNA synthesis kit (Roche, #4379012001) kit. Finally, the target gene to be determined is amplified and amplified using the PCR method using SensiFAST™ SYBR kit and primers (genomics, 100 nM) and treated with GAPDH as a reference gene and normalization.

The gene expression of immune-related factors analyzed above four millimeter-wave radiation sources versus control group (i.e. the same conditions but with no millimeter-wave exposure) were analyzed. Under each of the above conditions, the four kinds of millimeter-wave radiation mentioned above such as tumor necrosis factor (TNF-α) of THP-1 cells shown in FIG. 7, type II cyclooxygenase (COX-2) and the interleukin-6 (IL-6) gene shown in FIG. 9. The results of TNF-α and IL-6 expression induced by using P3 and P4 two millimeter wave radiation for 1.5 hours showed that the expression of TNF-α gene was 6-fold and nearly 9-fold higher than that of the control group respectively IL-6 gene expression than the control group nearly 17 times and 10 times with the significant effect. Furthermore, using the above four kinds of millimeter wave irradiation, the millimeter wave of P3 and P4 can achieve the induction of COX-2 at different radiation times. However, the millimeter wave of P2 at radiation for 2.5 hours can exhibit the effect of inhibiting the expression of COX-2. It is proved that the device of the present invention can generate millimeter waves with different waveforms by using millimeter wave parameters of different COXs. With the help of the time parameter, the performance of inducing or inhibiting −2 can be achieved. By penetrating the present invention and the method, (Macrophages, killer cells, T cells . . . , etc.) cytokines and immune-related factor performance of the purpose, and to control or/and change the body's immune system.

In addition, the cell culture solution collected at each time of the above experiment was used to test the concentration of each cytokine by ELISA, and the experimental result was presented in terms of how many pg of immune related factor contained per μgram in protein.

Male BALB/C mice (20-25 g) were experienced to administer intraperitoneally with LPS (30 μg/mouse in PBS) administered without millimeter-wave radiation and millimeter-wave radiation for 30 minutes, millimeters-wave radiation for 60 minutes, millimeter-wave radiation for 90 minutes, and millimeter-wave radiation for 150 minutes. The millimeter-wave radiation, the first mouse fixed in a plastic chamber, from top to bottom of the whole body radiation, radiation time before the day after the sacrifice of mice and mice obtained blood, isolated from the monocyte (macrophage) and plasma (plasma) for gene and protein performance analysis of mononuclear cells. Plasma was analyzed using a cytokine ELISA kit. It is also possible to show that the body of the animal radiated with the millimeter-wave radiation has a clear and under control or/and a modification of the immunity of the animal body.

The foregoing descriptions are merely exemplary embodiments of the present invention, and thus are not limitative of the scope of the present invention. In other words, the shapes, configurations, characteristics, methods, and it will be understood by one of ordinary skill in the art that various modifications can be made without departing from the spirit and the scope of the invention, as set forth in the appended claims.

What is claimed is:

1. An electromagnetic wave therapy device comprising:
A control unit providing a first control instruction;
A sensing unit, electrically connected to the control unit, for generating a sensing signal;
A memory unit, electrically connected to the control unit, for storing at least one data reference table, wherein the data reference table records a spectrum indication signal corresponding to the sensing signal;
A baseband unit electrically connected to the control unit for generating a corresponding therapeutic spectrum content according to the spectrum indicating signal; and
A radio frequency unit electrically connected to the control unit for emitting a millimeter-wave at a frequency between 56 GHz and 65 GHz and combining with the therapeutic spectrum content to become a millimeter-wave radiation source.

2. The electromagnetic wave therapy device according to claim 1, wherein the control unit, the baseband unit, the radio frequency unit and the memory unit are integrated in a single bio-chip to become a millimeter-wave therapeutic chip.

3. The electromagnetic wave therapy device according to claim 1, wherein the data reference table comprises an indication information, a plurality of cytokines parameters or an oxidative stress index parameter, and the sensing signal may correspond to the indication information, the plurality of cytokines or the oxidative stress index parameters and there is a corresponding of the spectrum indicating signal.

4. The electromagnetic wave therapy device according to claim 1, wherein the control unit is connected with a second control device, and the second control device can provide the data reference table, the spectrum indication signal or a second control instruction.

5. The electromagnetic wave therapy device according to claim 4, comprising:
A receiving unit, connected to the control unit and the second control device, for receiving the second control instruction or the data reference table; and
A transmitting unit, connected to the control unit and the second control device, for transmitting the sensing signal or the data reference table to the second control device.

6. The electromagnetic wave therapy device according to claim 1, comprising:
An antenna unit electrically connected to the radio frequency unit for focusing and transmitting the millimeter-wave radiation source.

7. The electromagnetic wave therapy device according to claim 1, comprising:
A display unit, electrically connected to the control unit, for displaying the sensing signal, the data reference table or the spectrum indicating signal;
An input interface electrically connected to the control unit; and
An expansion unit, electrically connected to the control unit, is a communication device or a data transmission device.

8. The electromagnetic wave therapy device according to claim 4, comprising an integrated database, electrically connected to the second control device for storing the data reference table.

9. The electromagnetic wave therapy device according to claim 1, wherein the data reference table comprises a timetable of emission parameter data, a waveform parameter data, a frequency parameter data, an energy parameter data, a pulse parameter data, an indication data, a sensing signal data, a plurality of cytokines parameter data, an oxidative stress index parameter, a spectrum indication signal and a combination of the above components.

10. An immunotherapy method using the electromagnetic wave therapy device according to claim 1, comprising following steps:
Confirming or input an indication of information;
Detecting an ambient temperature or a body temperature of a patient to generate the sensing signal;
A control unit reading a sensing signal and indication information and finding a corresponding spectrum indication signal in a data reference table;
Controlling a baseband unit to generate a therapeutic spectrum content according to a spectrum indication signal;
Controlling a radio frequency unit to generate a millimeter-wave with a frequency between 56 GHz and 65 GHz and making it a millimeter-wave radiation source in combination with the content of the therapeutic spectrum content; and
The millimeter-wave radiation source illuminating at least one indication area.

11. The immunotherapy method according to claim 10, comprising a medication with the indication.

12. The immunotherapy method according to claim 10, comprising:
Transmitting the sensing signal to a second control device;
The second control device further finding the corresponding spectrum indication signal from the sensing signal and the indication information; and
Transmitting the spectrum indication signal or the data reference table to the control unit.

13. The immunotherapy method according to claim 10, wherein the spectrum indication signal or data reference table is stored in the memory unit, and the data reference table comprises: a timetable of emission parameter data, a waveform parameter data, a frequency parameter data, an energy parameter data, a pulse parameter data, an indications data, a sensing signal data, a plurality of cytokine parameter data, an oxidative stress index parameter or spectral indication signal and other data.

14. The immunotherapy method according to claim 10, comprising:
The millimeter-wave radiation source being radiated with the indication area through the antenna unit.

15. The immunotherapy method according to claim 12, comprising:
The second control device generating a plurality of spectrum indication signals;
Transmitting the plurality of spectrum indication signals to the control unit and generating corresponding millimeter-wave illumination sources according to each of the spectrum indication signals; and
through the instruction of the second control device, radiating the patient each millimeter-wave radiation source to a corresponding different indication area.

16. The immunotherapy method according to claim 10, comprising:
Collecting and analyzing the radiation course record, treatment result, millimeter-wave sample data or sensing signal of the millimeter-wave radiation source, and then adjusting the spectrum indication signal.
